(19) United States
(12) Patent Application Publication
(10) Pub. No.: US 2024/0293626 A1
(43) Pub. Date: Sep. 5, 2024

(54) Safety Clip for Medical Injection Device

(71) Applicant: Becton Dickinson France, Le Pont-de-Claix (FR)

(72) Inventor: Nicolas Besson, Treffort (FR)

(21) Appl. No.: 18/566,312

(22) PCT Filed: Jun. 3, 2022
(86) PCT No.: PCT/EP2022/065183

§ 371 Date: Dec. 1, 2023

(30) Foreign Application Priority Data
Jun. 3, 2021 (EP) 21305745.8

(51) Int. Cl. A61M 5/315; A61M 5/32

(52) U.S. Cl. CPC A61M 5/31591; A61M 5/3202; A61M 2207/10

(57) ABSTRACT

Disclosed herein is a subassembly for a medical injection device and a medical injection device including the subassembly. The subassembly includes a first housing shell, a drive assembly configured to move a stopper of a syringe assembly, the drive assembly including a drive member, a plunger body, and a lever actuation member moveable between a locked position where actuation of the drive assembly is prevented and a released position where actuation of the drive assembly is allowed, the drive assembly configured to be received at least partially within the housing shell. The subassembly also includes a motor body, the motor body receiving at least a portion of the drive assembly therein and configured to be received at least partially within the housing shell, and a safety clip configured to couple to the motor body and to secure the lever actuation member in the locked position.

### CROSS-REFERENCE TO RELATED APPLICATION

The present invention claims priority to European Application No. 21305745.8, entitled “Safety Clip for Medical Injection Device”, filed Jun. 3, 2021, the entire disclosure of which is hereby incorporated by reference in its entirety.

### BACKGROUND OF THE INVENTION

### Field of the Invention

The present invention relates generally to safety clips for medical injection devices, and, in particular embodiments or aspects, to safety clips that can be attached to a medical injection device during assembly to prevent accidental firing.

### Description of Related Art

Medical injection devices, in particular autoinjectors, often are provided as a plurality of separate components and/or subassemblies. These components and/or subassemblies are then assembled by the customers, for example pharmaceutical manufacturers, with a pre-filled syringe of a given medicament.

However, issues can arise during assembly, whereby a pre-cocked drive assembly can accidentally be actuated, rendering the injection device unusable. While locking arrangements are known, such arrangements typically require a step of attaching a locking component during assembly and removing the same prior to a final stage of assembly, which can slow the pace of assembly, and, if accidentally left in place in the device, can render the injection device unusable. Accordingly, there is a need in the art for a safety device that prevents accidental firing of a medical injection device during assembly, but that does not require separate coupling and decoupling steps.

### SUMMARY OF THE INVENTION

Provided herein is a subassembly for a medical injection device including a first housing shell, a drive assembly configured to move a stopper of a syringe assembly, the drive assembly having a drive member, a plunger body, and a lever actuation member moveable between a locked position where actuation of the drive assembly is prevented and a released position where actuation of the drive assembly is allowed, the drive assembly configured to be received at least partially within the housing shell, a motor body, the motor body receiving at least a portion of the drive assembly therein and configured to be received at least partially within the housing shell, and a safety clip configured to couple to the motor body and to secure the lever actuation member in the locked position.

Also provided herein is a medical injection device including a first subassembly for a medical injection device including a first housing shell, a drive assembly configured to move a stopper of a syringe assembly, the drive assembly having a drive member, a plunger body, and a lever actuation member moveable between a locked position where actuation of the drive assembly is prevented and a released position where actuation of the drive assembly is allowed, the drive assembly configured to be received at least partially within the housing shell, a motor body, the motor body receiving at least a portion of the drive assembly therein and configured to be received at least partially within the housing shell, and a safety clip configured to couple to the motor body and to secure the lever actuation member in the locked position, and a second subassembly including a cap, a needle cover, a syringe holder configured to receive a syringe assembly, a cassette body, and a second housing shell, the cap receiving at least a portion of the needle cover, wherein upon assembly of the first subassembly to the second subassembly, the safety clip is displaced, allowing the lever actuation member to move to the released position.

Also provided herein is a method of assembling a medical injection device including steps of moving a first subassembly into engagement with a first housing shell, the first subassembly including a drive assembly configured to move a stopper of a syringe assembly, the drive assembly having a drive member, a plunger body, and a lever actuation member moveable between a locked position where actuation of the drive assembly is prevented and a released position where actuation of the drive assembly is allowed, the drive assembly configured to be received at least partially within the first housing shell, a motor body, the motor body receiving at least a portion of the drive assembly therein and configured to be received at least partially within the first housing shell, a safety clip configured to couple to the motor body and to secure the lever actuation member in the locked position, wherein moving the first subassembly into engagement with the first housing shell causes the safety clip to move to a first position in which the lever actuation member is secured in the locked positon.

Further embodiments or aspects of the present invention are provided in the following numbered clauses:

Clause 1. A subassembly for a medical injection device comprising: a first housing shell; a drive assembly configured to move a stopper of a syringe assembly, the drive assembly comprising a drive member, a plunger body, and a lever actuation member moveable between a locked position where actuation of the drive assembly is prevented and a released position where actuation of the drive assembly is allowed, the drive assembly configured to be received at least partially within the housing shell; a motor body, the motor body receiving at least a portion of the drive assembly therein and configured to be received at least partially within the housing shell; and a safety clip configured to couple to the motor body and to secure the lever actuation member in the locked position.

Clause 2. The subassembly according to clause 1, wherein the safety clip is configured to secure the lever actuation member in the locked position when the drive assembly and motor body are received within the housing shell.

Clause 3. The subassembly according to clause 1 or clause 2, wherein the safety clip comprises a proximal end and a distal end, a first member arranged at the proximal end, second and third members extending distally from the first member, and a fourth member connected to the second and third members.

Clause 4. The subassembly according to clause 3, wherein the first member is substantially u-shaped and comprises a base and a plurality of arms extending radially substantially perpendicular to the base.

Clause 5. The subassembly according to clause 4, wherein the plurality of arms comprise a first end connected to the base and a second end spaced from the first end, the second end comprising an angled surface.

Clause 6. The subassembly according to clause 5, wherein the angled surface is complimentary to at least a portion of the lever actuation member.

Clause 7. The subassembly according to any of clauses 3-6, wherein the second and third members are arranged substantially perpendicular to the first member.

Clause 8. The subassembly according to any of clauses 3-7, wherein the second member and third member are arranged substantially parallel to each other.

Clause 9. The subassembly according to any of clauses 3-8, wherein the fourth member is arranged between the proximal end and the distal end of the safety clip.

Clause 10. The subassembly according to any of clauses 3-9, wherein the fourth member is moveable between a first position in which the fourth member is substantially linear and a second position in which the fourth member is angled.

Clause 11. The subassembly according to clause 10, wherein in the first position, the fourth member is parallel to the first member.

Clause 12. The subassembly according to clause 10 or clause 11, wherein the fourth member is in the second positon when the drive assembly and the motor body are received within the housing shell.

Clause 13. A medical injection device comprising: a first subassembly comprising the subassembly according to any of clauses 1-12; and a second subassembly comprising: a cap, a needle cover, a syringe holder configured to receive a syringe assembly, a cassette body, and a second housing shell, the cap receiving at least a portion of the needle cover, wherein upon assembly of the first subassembly to the second subassembly, the safety clip is displaced, allowing the lever actuation member to move to the released position.

Clause 14. A method of assembling a medical injection device comprising: moving a first subassembly into engagement with a first housing shell, the first subassembly comprising: a drive assembly configured to move a stopper of a syringe assembly, the drive assembly comprising a drive member, a plunger body, and a lever actuation member moveable between a locked position where actuation of the drive assembly is prevented and a released position where actuation of the drive assembly is allowed, the drive assembly configured to be received at least partially within the first housing shell; a motor body, the motor body receiving at least a portion of the drive assembly therein and configured to be received at least partially within the first housing shell; and a safety clip configured to couple to the motor body and to secure the lever actuation member in the locked position, wherein moving the first subassembly into engagement with the first housing shell causes the safety clip to move to a first position in which the lever actuation member is secured in the locked positon.

Clause 15. The method according to clause 14, further comprising; moving the first subassembly into engagement with a second subassembly, the second subassembly comprising: a cap, a needle cover, a syringe holder configured to receive a syringe assembly, a cassette body, and a lower housing shell, wherein moving the first subassembly into engagement with the second subassembly causes the safety clip to move to a second positon in which the lever actuation member is free to move to the released position.

### BRIEF DESCRIPTION OF THE DRAWINGS

FIG. 1 is a side view of a medical injection device according to non-limiting embodiments or aspects;

FIG. 2 is a cross-sectional view through plane 2-2 of FIG. 1 of a medical injection device according to non-limiting embodiments or aspects;

FIG. 3 is a perspective view of a medical injection device according to non-limiting embodiments or aspects;

FIG. 4 is an exploded view of a medical injection device according to non-limiting embodiments or aspects;

FIG. 5 is a perspective view of a safety device for a medical injection device according to non-limiting embodiments or aspects;

FIG. 6 is a perspective view of a medical injection device according to non-limiting embodiments or aspects;

FIG. 7 is a partial perspective view of internal components of a medical injection device according to non-limiting embodiments or aspects;

FIG. 8 is a partial side view of internal components of a medical injection device according to non-limiting embodiments or aspects;

FIG. 9 is a partial side view of internal components of a medical injection device according to non-limiting embodiments or aspects;

FIG. 10 is a partial perspective view of a medical injection device according to non-limiting embodiments or aspects;

FIG. 11 is a partial side view of internal components of a medical injection device according to non-limiting embodiments or aspects;

FIG. 12 is a partial side view of internal components of a medical injection device

according to non-limiting embodiments or aspects;

FIG. 13 is a partial side view of internal components of a medical injection device according to non-limiting embodiments or aspects;

FIG. 14 is a partial side view of internal components of a medical injection device according to non-limiting embodiments or aspects;

FIG. 15 is a partial perspective view of a medical injection device according to non-limiting embodiments or aspects; and

FIG. 16 is a partial perspective view of a medical injection device according to non-limiting embodiments or aspects.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

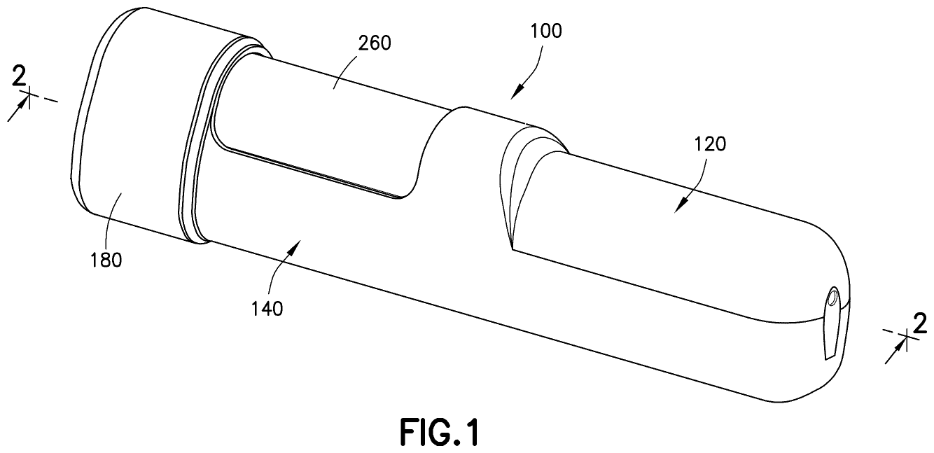

Referring to FIGS. 1-3, a medical injection device 100 according to one embodiment or aspect includes a first subassembly 120, a second subassembly 140, and a syringe assembly 160. The first subassembly 120 includes a drive assembly 300, a motor body 320, a lever actuation member 340, and an upper housing shell 360. The second subassembly 140 includes a cap 180 having a retainer 200, a needle cover 220, a syringe holder 240, a cassette body 260, and a lower housing shell 280. The syringe assembly 160 is received by the syringe holder 240 and includes a barrel 162, a stopper 166, a cannula 164, and a rigid needle shield (RNS) 168. Although an RNS is utilized, other suitable needle shield arrangements may be utilized. The lower housing shell 280, the cassette body 260, and the upper housing shell 360 generally form a housing for receiving the various components of the medical injection device 100, although other suitable housing arrangements may be utilized. The medical injection device 100 may be an auto-injector, although the features described herein may be incorporated into other suitable medical injection devices.

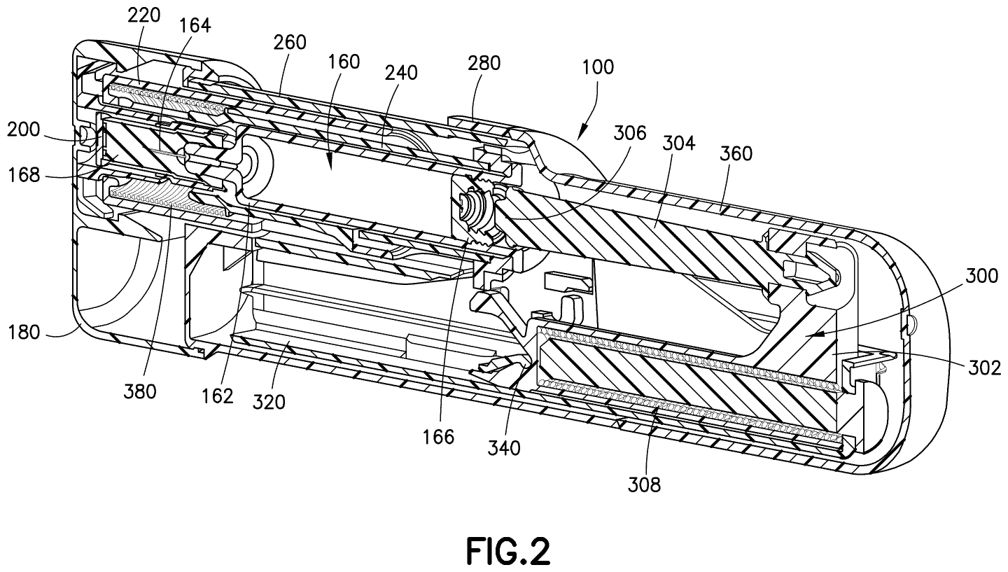

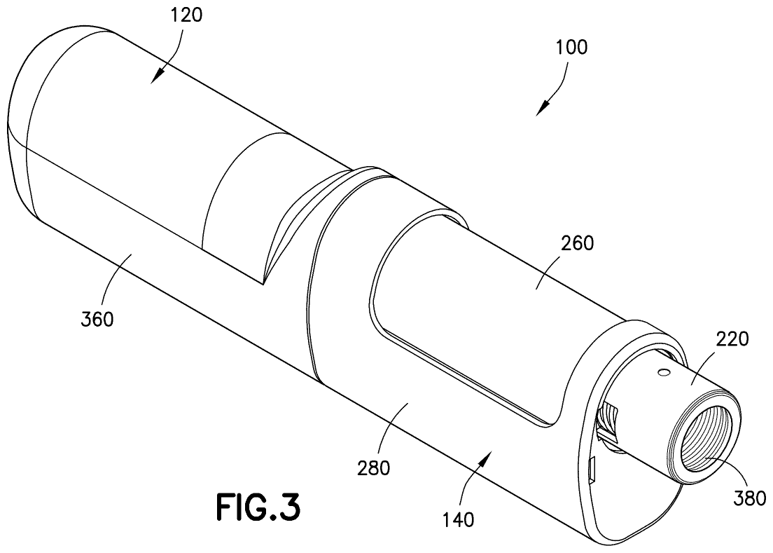

The medical injection device 100 is configured to automatically deliver a dose of medicament from the syringe assembly 160 to a patient upon actuation of the medical injection device 100. More specifically, upon actuation of the medical injection device 100, the drive assembly 300 is configured to engage the stopper 166 of the syringe assembly 160, displace the syringe assembly 160 such that the cannula 164 pierces the skin of the patient, and displace the stopper 166 within the barrel 162 of the syringe assembly 160 to deliver the medicament within the barrel 162. The medical injection device 100 includes a storage position (FIGS. 1 and 2), a pre-use position (FIG. 3), and an actuation position, an injection, and a post-use position (not shown). When assembled and ready for use, the needle cover 220 is configured to shield the cannula 164 of the syringe assembly 160 from the patient. In particular, the needle cover 220 is moveable between a pre-use position, an actuation position, and a post-use positon, with a spring 380 biasing the needle cover 220 towards the pre-use position and the post-use position. The spring 380 is positioned between the needle cover 220 and the syringe holder 240, although other suitable arrangements may be utilized. The lever actuation member 340 is moveable between a locked position where movement of the drive assembly 300 is prevented and a released position where movement of the drive assembly 300 is allowed. More specifically, the lever actuation member 340 is rotatable about a rotation axis between the locked position and the released position. When the lever actuation member 340 is in the locked position, the lever actuation member 340 is engaged with the motor body 320 and the drive assembly 300 to prevent movement of the drive assembly 300. When the lever actuation member 340 is in the released position, the lever actuation member 340 is disengaged from the motor body 320 thereby allowing movement of the drive assembly 340 toward the syringe assembly 160. The rotation axis of the lever actuation member 340 extends perpendicular to a longitudinal axis of the medical injection device 100, although other suitable arrangements may be utilized.

As noted above, the lever actuation member 340, when in the locked positon, prevents movement of the drive assembly 300. During assembly, before or during the connection of the first subassembly 120 to the second subassembly 140, the lever actuation member 340 could inadvertently be moved to the released position, resulting in unwanted firing of the drive assembly 300. Safety clip 400 prevents this transition of the lever actuation member during assembly of the medical injection device 100.

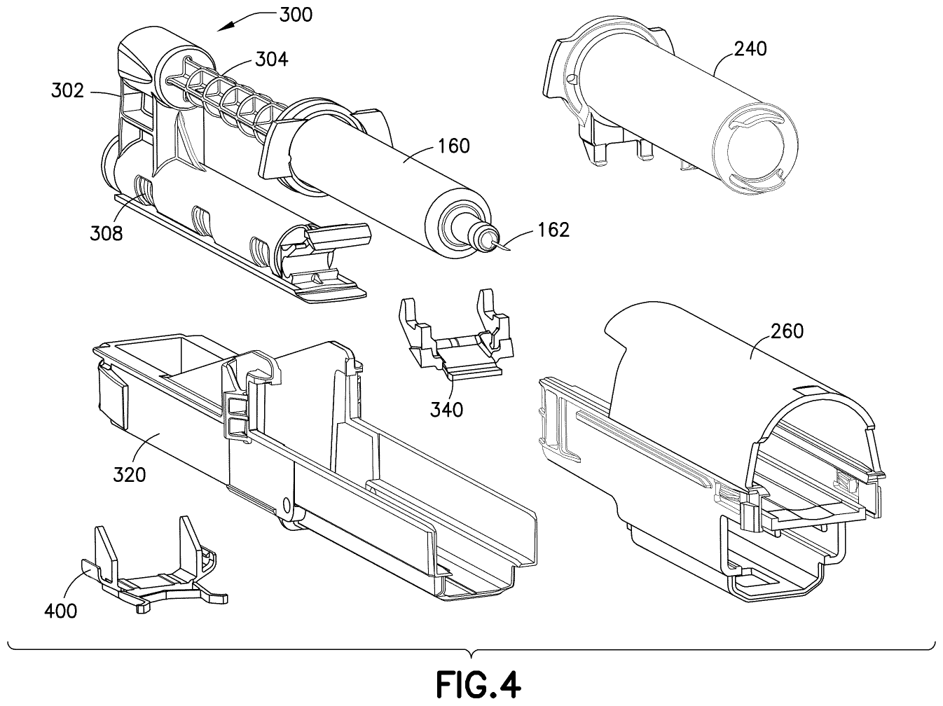

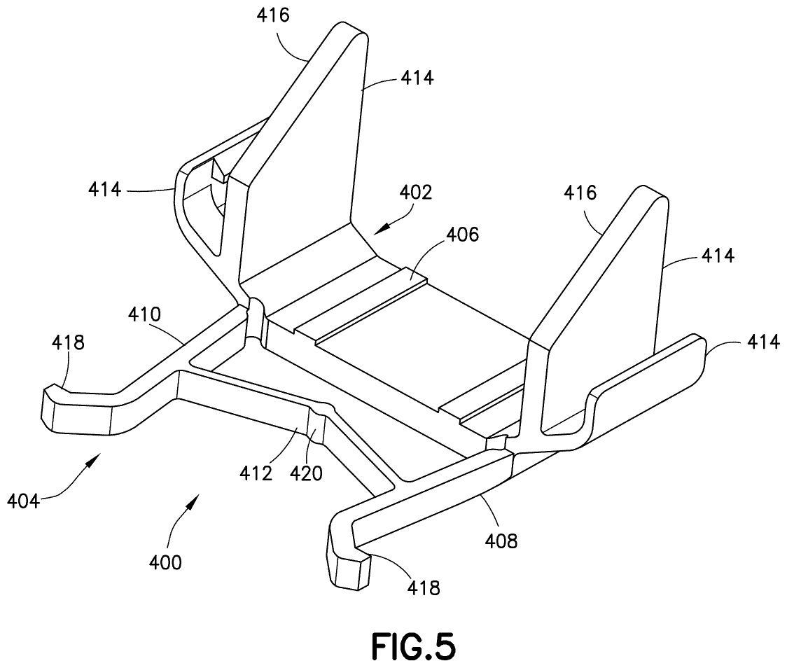

Turning to FIG. 4, an exploded view of a medical injection device 100 is shown. Medical injection device 100 includes previously-described components, such as syringe assembly 160, syringe holder 240, cassette body 260, drive assembly 300, and motor body 320. Medical injection device 100 also includes safety clip 400. FIG. 5 shows a non-limiting embodiment or aspects of safety clip 400. In non-limiting embodiments or aspects, safety clip 400 includes a body having a proximal end 402 and distal end 404. At proximal end 402, a first member 406 can be arranged. While shown in FIG. 5 as a generally u-shaped member (including one or more arms 414 extending generally perpendicular in a radial direction therefrom), first member 406 can assume any useful configuration. One or more arms 414 can also include one or more angled surfaces 416. One or more angled surfaces 416 can be configured to interact with lever actuation member 340, to prevent rotation thereof, and, thus, prevent actuation of drive assembly 300.

In non-limiting embodiments or aspects, a first subassembly for a medical injection device 100 is shown. First subassembly 120 includes a drive assembly 300 (including plunger body 302, and plunger rod 304), a motor body 320, a lever actuation member 340, a first housing shell 360, and safety clip 400. As described above, the lever actuation member 340 can assume two or more positions, a locked position where actuation of the drive assembly 300 is prevented and a released position where actuation of the drive assembly 300 is allowed. Drive assembly 300 is received at least partially within motor body 320, and both motor body 320 and drive assembly 300 are received at least partially within first housing shell 360. As described herein, safety clip 400 is configured to couple to motor body 320 and to secure the lever actuation member 340 in the locked position.

Safety clip 400 can also include second member 408 and/or third member 410. Second member 408 and third member 410 can extend generally perpendicular to a long axis of first member 406, for example as shown in FIG. 5. At a distal end 404, second member 408 and third member 410 can include one or more engagement portions 418 for engaging a housing shell of medical injection device 100, such as upper housing shell 360. Safety clip 400 can also include, in non-limiting embodiments or aspects, fourth member 412. Fourth member 412 can be arranged generally parallel to a long axis of first member 406, and can be positioned between first member 406 and distal end 404. Fourth member 412 can be arranged generally perpendicular to second member 408 and third member 410. Fourth member 412 can be configured to be flexible, allowing second member 408 and third member 410 to be displaced towards one another, while fourth member 412 flexes towards first member 406 or towards distal end 404 of safety clip 400 about flex point 420. During flexure, fourth member 412 can move from a first, generally linear positon, to a second, generally non-linear position, such as an angled arrangement.

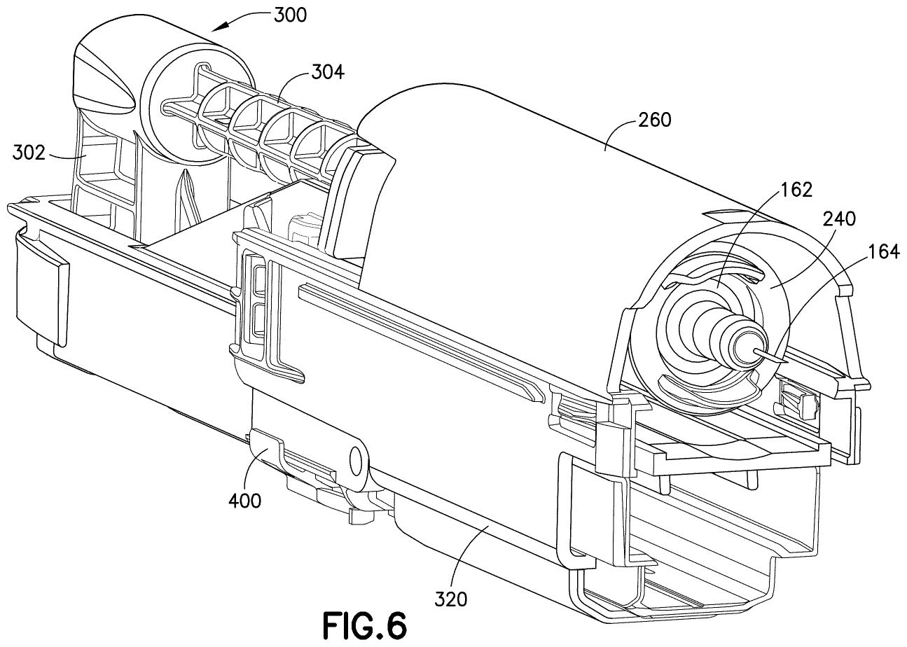

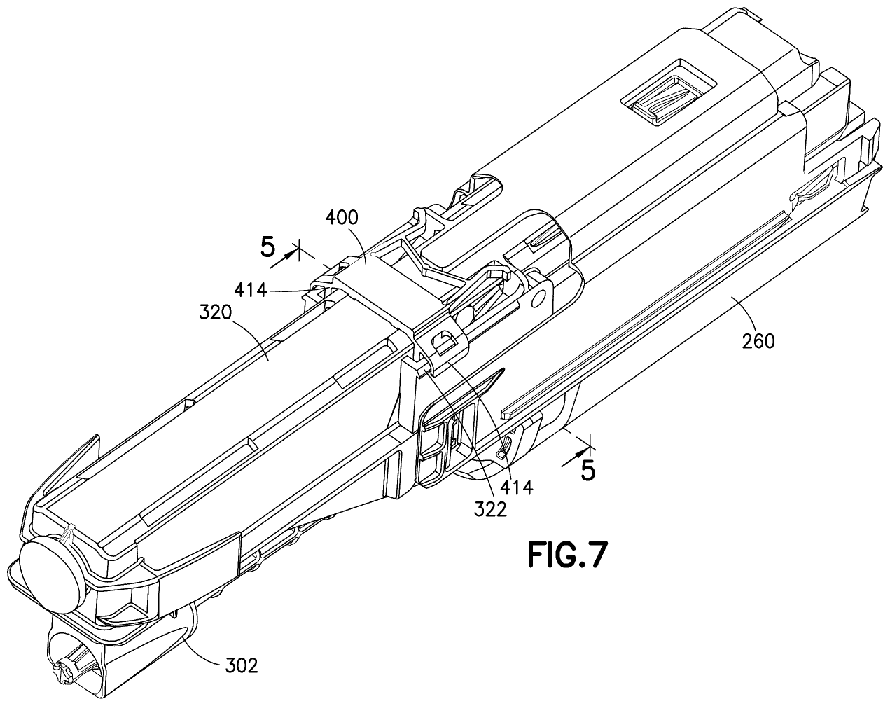

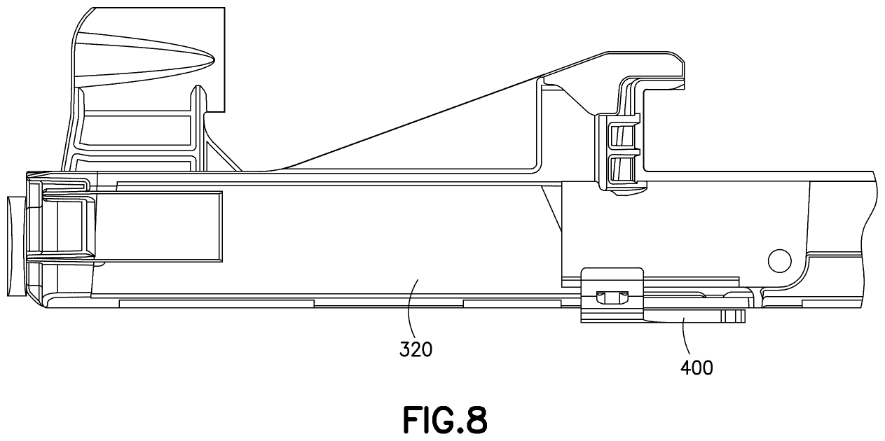

Turning to FIGS. 6-8, shown are various views of medical injection device 100 without housing shell portions, to show the interaction between safety clip 400 and motor body 320. Safety clip 400 can be configured to interact with motor body 320 in any suitable manner. As shown in FIG. 7, one or more arms 414 can be configured to interact with one or more cooperating elements on motor body 320. For example safety clip 400 can be formed of a resilient material, such as a resilient plastic or metal, and one or more arms 414 can be resiliently biased to a radially-inward position. Safety clip 400 can then be engaged with motor body 320, such that one or more arms 414 snap or clip into engagement with a cooperating element on motor body 320, such as rail 322.

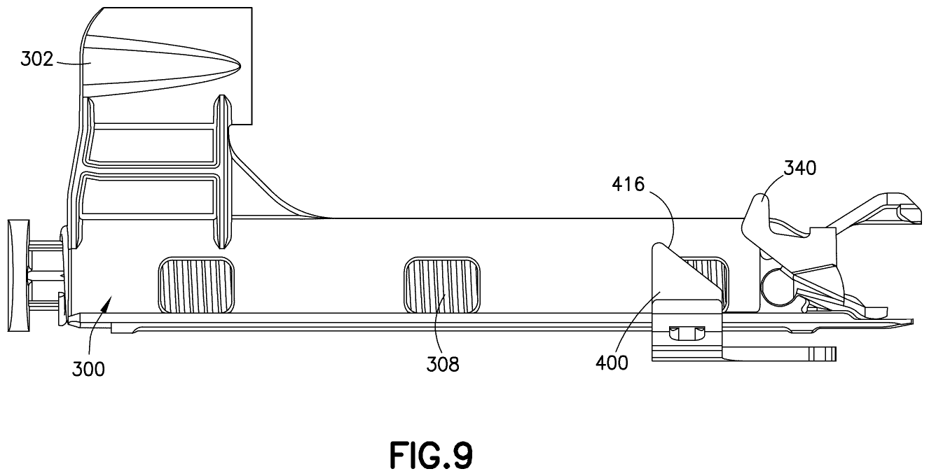

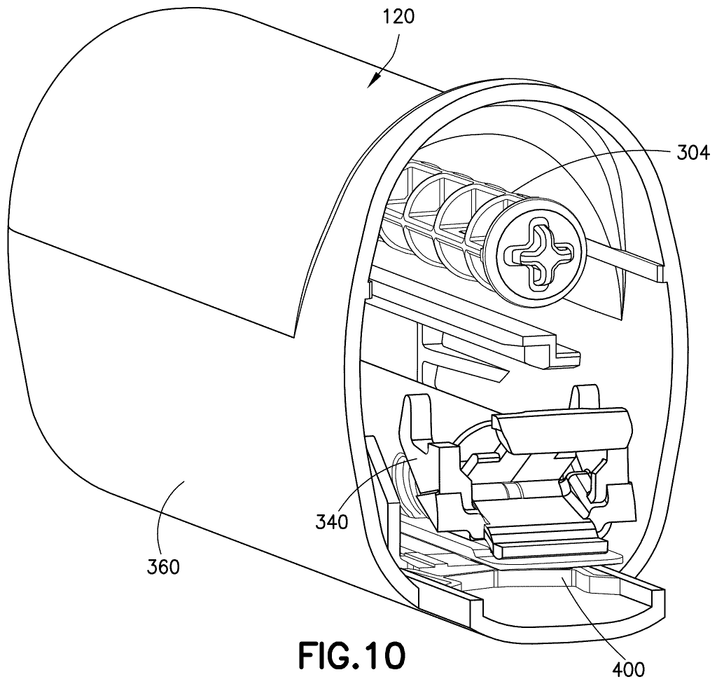

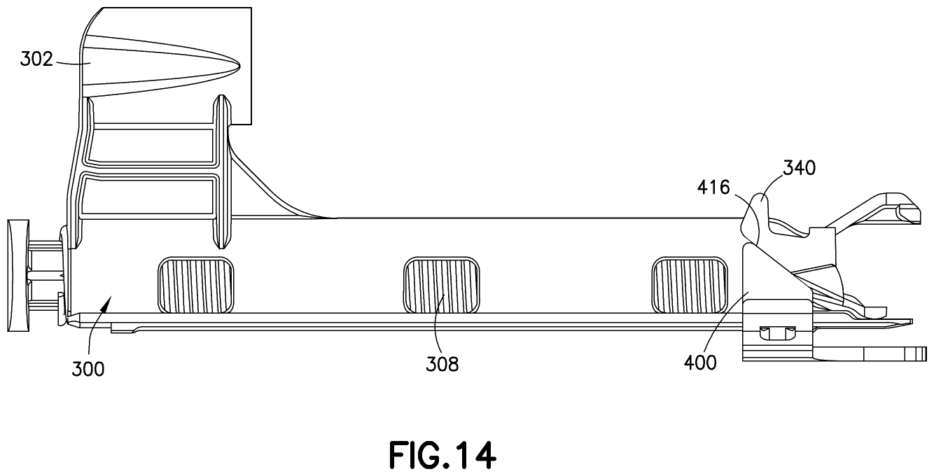

Turning to FIGS. 9-15, shown are select, isolated components of first subassembly 120 during a process of assembling medical injection device 100. Those of skill in the art will appreciate that fully assembled medical injection device 100 includes the components listed and described above, and that a number of the components are not shown in FIGS. 9-15 to allow for visualization of the interaction of safety clip 400 with motor body 320, lever actuation member 340, and upper housing shell 360. Turning to FIG. 9, shown are motor body 320, lever actuation member 340, and safety clip 400 in a position prior to placement of upper housing shell 360 on first subassembly 120. Safety clip 400 is engaged with motor body 320, but is spaced from lever actuation member 340. FIG. 10 shows initial placement of upper housing shell 360 on remaining components of first subassembly 120. As can be seen in FIG. 10, safety clip 400 remains spaced from lever actuation member 340.

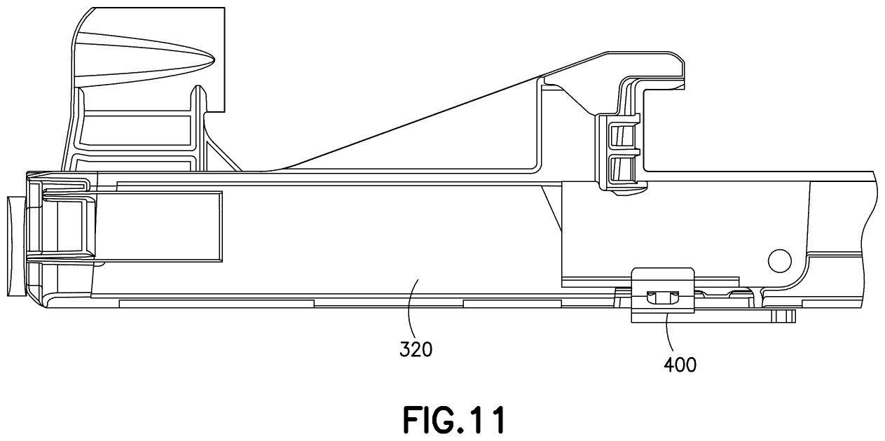

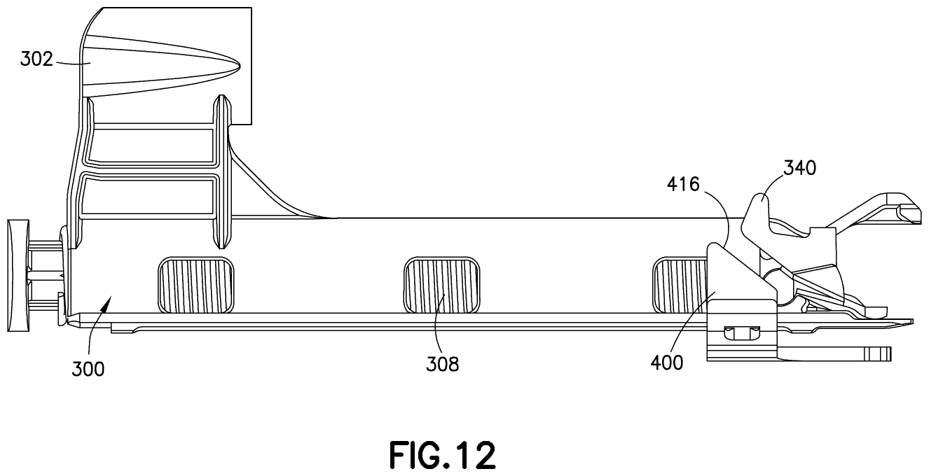

Turning to FIGS. 11 and 12, shown is the interaction of safety clip 400 and motor body 320 as upper housing shell (not shown) is further engaged with remaining components of first subassembly 120. In comparison with FIG. 9, safety clip 400 has been displaced from the left (an upper position relative to the motor body 320) to the right (a lower position relative to motor body 320), though safety clip 400 remains spaced from lever actuation member 340.

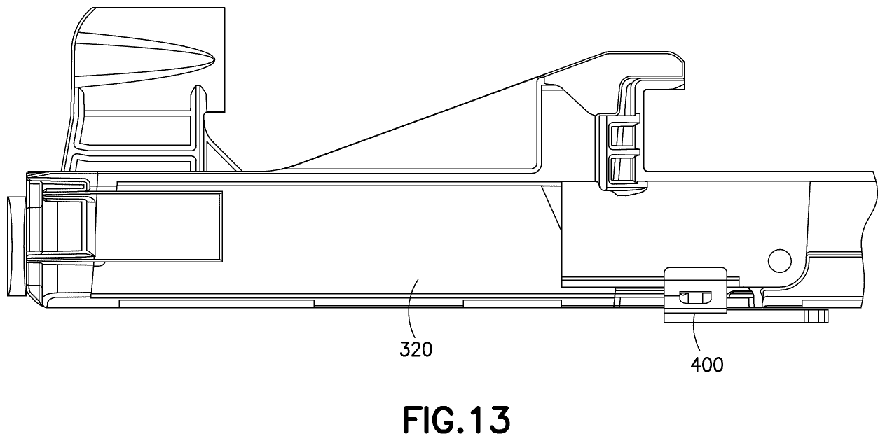

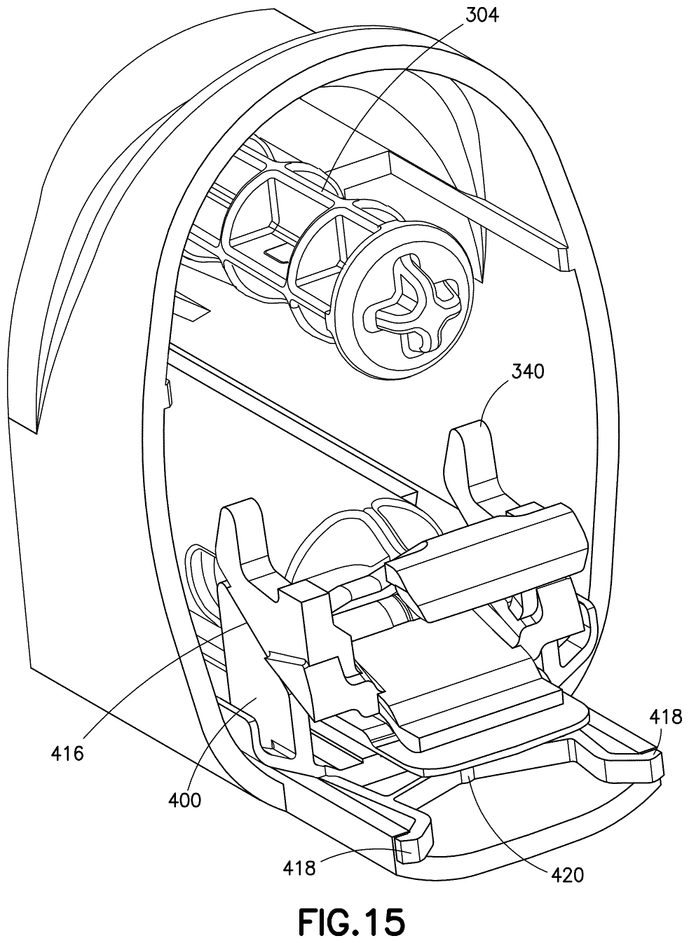

Turning to FIGS. 13-15, shown is the interaction of safety clip 400 and motor body 320 as upper housing shell (not shown) is fully engaged with remaining components of first subassembly 120. In comparison with FIGS. 9, 11, and 12, safety clip 400 has been displaced from the left (an upper position relative to the motor body 320) to the right (a lower position relative to motor body 320). In this position, angled surface 416 of safety clip 400 is engaged with lever actuation member 340, and thus, prevents rotation of lever actuation member from the locked positon to the released position. As shown in FIG. 15, lever actuation member 340 cannot rotate and force safety clip 400 to be displaced radially outward, due to the confinement of safety clip 400 within upper housing shell 360. In addition, lever actuation member 340 cannot rotate and force safety clip 400 to be displaced longitudinally, due to the interaction of engagement portions 418 with upper housing shell 360. Engagement portions 418 at distal end 404 of safety clip 400 can snap into engagement with upper housing shell 360 due to resilient bias of the material from which safety clip 400 is formed and/or force provided by fourth member 412 being biased to return to a generally linear position (about flex point 420).

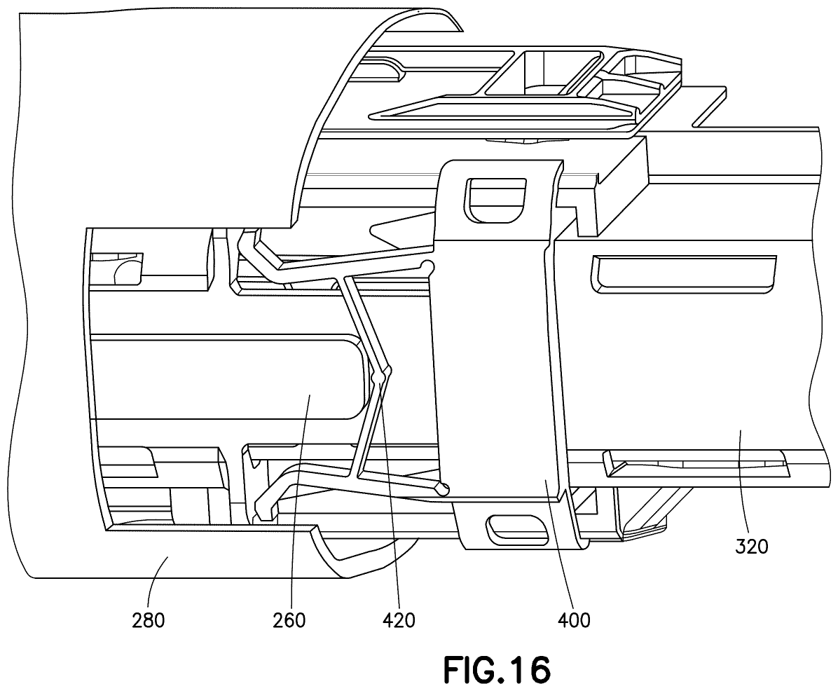

Turning to FIG. 16, interaction of cassette body 260 and safety clip 400 during final assembly of medical injection device 100 is shown. During final assembly of medical injection device 100, first subassembly 120 (including drive assembly 300, plunger body 302, plunger rod 304, motor body 320, lever actuation member 340, first housing shell 360, and safety clip 400) is placed onto a second subassembly 140 (including cap 180 having a retainer 200, needle cover 220, syringe holder 240, cassette body 260, and lower housing shell 280). As first subassembly 120 is moved into engagement with second subassembly 140, cassette body 260 contacts safety clip 400. In non-limiting embodiments or aspects, cassette body 260 contacts fourth member 412, in some embodiments or aspects at or about flex point 420, and as first subassembly 120 is further engaged with second subassembly 140, interaction between cassette body 260 and safety clip 400 causes second member 408 and third member 410 to be deflected inwards, causing engagement portions 418 to fall out of engagement with upper housing shell 360. As engagement portions 418 fall out of engagement with upper housing shell 360, continued engagement of cassette body 260 with safety clip 400 and continued placement of first subassembly 120 on second subassembly 140 causes safety clip 400 to be displaced to a position, for example such as that shown in FIG. 9, spaced from lever actuation member 340. Thus, when fully assembled, medical injection device 100 can be actuated, because safety clip 400 is no longer engaged with lever actuation member 340, and no longer prevents rotation of lever actuation member 340 from a locked positon to a released position.

Although the invention has been described in detail for the purpose of illustration based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A subassembly for a medical injection device comprising:
a first housing shell;
a drive assembly configured to move a stopper of a syringe assembly, the drive assembly comprising a drive member, a plunger body, and a lever actuation member moveable between a locked position where actuation of the drive assembly is prevented and a released position where actuation of the drive assembly is allowed, the drive assembly configured to be received at least partially within the housing shell;
a motor body, the motor body receiving at least a portion of the drive assembly therein and configured to be received at least partially within the housing shell; and
a safety clip configured to couple to the motor body and to secure the lever actuation member in the locked position.

2. The subassembly according to claim 1, wherein the safety clip is configured to secure the lever actuation member in the locked position when the drive assembly and motor body are received within the housing shell.

3. The subassembly according to claim 1, wherein the safety clip comprises a proximal end and a distal end, a first member arranged at the proximal end, second and third members extending distally from the first member, and a fourth member connected to the second and third members.

4. The subassembly according to claim 3, wherein the first member is substantially u-shaped and comprises a base and a plurality of arms extending radially substantially perpendicular to the base.

5. The subassembly according to claim 4, wherein the plurality of arms comprise a first end connected to the base and a second end spaced from the first end, the second end comprising an angled surface.

6. The subassembly according to claim 5, wherein the angled surface is complimentary to at least a portion of the lever actuation member.

7. The subassembly according to claim 3, wherein the second and third members are arranged substantially perpendicular to the first member.

8. The subassembly according to claim 3, wherein the second member and third member are arranged substantially parallel to each other.

9. The subassembly according to claim 3, wherein the fourth member is arranged between the proximal end and the distal end of the safety clip.

10. The subassembly according to claim 3, wherein the fourth member is moveable between a first position in which the fourth member is substantially linear and a second position in which the fourth member is angled.

11. The subassembly according to claim 10, wherein in the first position, the fourth member is parallel to the first member.

12. The subassembly according to claim 10, wherein the fourth member is in the second positon when the drive assembly and the motor body are received within the housing shell.

13. A medical injection device comprising:
a first subassembly comprising:
a first housing shell,
a drive assembly configured to move a stopper of a syringe assembly, the drive assembly comprising a drive member, a plunger body, and a lever actuation member moveable between a locked position where actuation of the drive assembly is prevented and a release position where actuation of the drive assembly is allowed, the drive assembly configured to be received at least partially within the housing shell;
a motor body, the motor body receiving at least a portion of the drive assembly therein and configured to be received at least partially within the housing shell; and
a safety clip configured to couple to the motor body and to secure the lever actuation member in the locked position,
and
a second subassembly comprising:
a cap, a needle cover, a syringe holder configured to receive a syringe assembly, a cassette body, and a second housing shell, the cap receiving at least a portion of the needle cover,
wherein upon assembly of the first subassembly to the second subassembly, the safety clip is displaced, allowing the lever actuation member to move to the released position.

14. A method of assembling a medical injection device comprising:
moving a first subassembly into engagement with a first housing shell, the first subassembly comprising:
a drive assembly configured to move a stopper of a syringe assembly, the drive assembly comprising a drive member, a plunger body, and a lever actuation member moveable between a locked position where actuation of the drive assembly is prevented and a released position where actuation of the drive assembly is allowed, the drive assembly configured to be received at least partially within the first housing shell;
a motor body, the motor body receiving at least a portion of the drive assembly therein and configured to be received at least partially within the first housing shell; and
a safety clip configured to couple to the motor body and to secure the lever actuation member in the locked position,
wherein moving the first subassembly into engagement with the first housing shell causes the safety clip to move to a first position in which the lever actuation member is secured in the locked positon.

15. The method according to claim 14, further comprising;
moving the first subassembly into engagement with a second subassembly, the second subassembly comprising:
a cap, a needle cover, a syringe holder configured to receive a syringe assembly, a cassette body, and a lower housing shell,
wherein moving the first subassembly into engagement with the second subassembly causes the safety clip to move to a second positon in which the lever actuation member is free to move to the released position.

16. The medical injection device according to claim 13, wherein the safety clip is configured to secure the lever actuation member in the locked position when the drive assembly and motor body are received within the housing shell.

17. The medical injection device according to claim 13, wherein the safety clip comprises a proximal end and a distal end, a first member arranged at the proximal end, second and third members extending distally from the first member, and a fourth member connected to the second and third members.

18. The medical injection device according to claim 13, wherein the first member is substantially u-shaped and comprises a base and a plurality of arms extending radially substantially perpendicular to the base.

19. The medical injection device according to claim 18, wherein the plurality of arms comprise a first end connected to the base and a second end spaced from the first end, the second end comprising an angled surface.

20. The medical injection device according to claim 19, wherein the angled surface is complimentary to at least a portion of the lever actuation member.
